(19) United States
(12) Patent Application Publication
(10) Pub. No.: US 2026/0157862 A1
(43) Pub. Date: Jun. 11, 2026

(54) PROCESSED BONE RETAINER

(71) Applicant: NISSIN FULFIL CO., LTD, Kyoto (JP)

(72) Inventors: Ryosuke YAMAZOE, Kyoto (JP); Shuhei MURAOKA, Kyoto (JP); Hidekazu TSUJI, Kyoto (JP); Kenji INOUE, Kyoto (JP); Koji TANAKA, Kyoto (JP); Akira NISHIKOHRI, Kyoto (JP)

(21) Appl. No.: 18/707,343

(22) PCT Filed: Nov. 4, 2022
(86) PCT No.: PCT/JP2022/041229

§ 371 Date: May 3, 2024

(30) Foreign Application Priority Data
Nov. 5, 2021 (JP) 2021-180966

(51) Int. Cl. A61F 2/46

(52) U.S. Cl. CPC A61F 2/4644; A61F 2002/4645

(57) ABSTRACT

CLEAN COPY OF THE ABSTRACT A retainer for a bone to be processed includes: a base table including: an inserter that includes a rod shape, and is inserted into the through-hole of a bone to be processed; and a supporter that includes a rod shape, and is linked to the inserter at one end, and of which the area of a cross section that is orthogonal to a central axis direction is larger than the area of a cross section of the through-hole, the cross section being orthogonal to the central axis direction; and a nip member that includes a screw hole and nips, together with the supporter, the bone to be processed in the state of being attached threadedly to the inserter and abutting on the bone to be processed.

### CROSS-REFERENCE TO RELATED APPLICATIONS

This application is a national stage application, filed under 35 U.S.C. § 371, of International Patent Application No. PCT/JP 2022/041229 filed Nov. 4, 2022, which claims the benefit of Japanese Patent Application No. JP 2021-180966 filed Nov. 5, 2021, the entire disclosures of which are incorporated herein by reference for all purposes.

### TECHNICAL FIELD

The present disclosure relates to a retainer for a bone to be processed.

### BACKGROUND ART

Techniques to produce screws that are used in osteosynthesis for treating avulsion fracture and the like, by cutting bone chips collected from the bones of patients, have been proposed (see, for example, Patent Literature 1). In such cases, cutting processing is performed by allowing cutting tools to abut on bone chips in a state in which bone chips are gripped by chucking devices.

For example, workpiece retaining devices that subject workpieces to centering-chucking by a plurality of chuck nails disposed at equal angles have been proposed as the chucking devices that are used in the kinds of cutting processing (see, for example, Patent Literature 2).

CITATION LIST

### Patent Literature

Patent Literature 1: Unexamined Japanese Patent Application Publication No. 2006-239825
Patent Literature 2: Unexamined Japanese Patent Application Publication No. 2012-228766

### SUMMARY OF INVENTION

### Technical Problem

An amount as small as possible of a bone chip that is used for producing a screw is requested from the viewpoint of reducing a burden on a patient. However, in the case of using a workpiece retaining device described in Patent Literature 2, a bone chip of which the size enables security of a portion that is gripped by at least a plurality of chuck nails is needed, and therefore, the amount of a collected bone is increased corresponding thereto, and a burden on a patient may be increased.

The present disclosure was made under such circumstances with an objective to provide a retainer for a bone to be processed that can reduce a burden on a patient.

### Solution to Problem

In order to achieve the objective described above, a retainer for a bone to be processed according to the present disclosure is a retainer for a bone to be processed that retains a bone to be processed that has a long column shape and in which a through-hole that extends along a longitudinal direction is formed, the retainer for a bone to be processed including:
a base table including: an inserter that has a rod shape and a first male screw that is formed on a side wall along a longitudinal direction, and is inserted into the through-hole; and a supporter that has a rod shape, is linked to the inserter at one end in a longitudinal direction, and supports the bone to be processed, and of which an area of a cross section that is orthogonal to the longitudinal direction is larger than an area of a cross section of the through-hole, the cross section being orthogonal to the longitudinal direction of the bone to be processed; and
a nip member that has a first screw hole on an inner wall of which a first female screw that is threadedly engaged with the first male screw is formed, and nips, together with the one end of the supporter, the bone to be processed in a state of being attached threadedly to the inserter and abutting on the bone to be processed.

### Advantageous Effects of Invention

In accordance with the present disclosure, a base table includes: an inserter that is inserted into the through-hole of a bone to be processed; and a supporter of which the area of a cross section that is orthogonal to in a longitudinal direction is larger than the area of a cross section of the through-hole of the bone to be processed, the cross section being orthogonal to the longitudinal direction of the bone to be processed. Moreover, a nip member nips, together with the supporter, the bone to be processed in the state of being attached threadedly to the inserter of the base table and abutting on the bone to be processed. Thus, the bone to be processed can be firmly retained without gripping the side wall of the bone to be processed, and therefore, the amount of a bone to be processed that is required to be collected from a patient can be reduced. Therefore, a burden on the patient can be reduced.

### BRIEF DESCRIPTION OF DRAWINGS

FIG. 1 is a schematic view of a cutting-processing device according to Embodiment 1 of the present disclosure;

FIG. 2 is a perspective view of a retainer for a bone to be processed and a scroll chuck according to Embodiment 1;

FIG. 3 is a cross sectional view of the retainer for a bone to be processed and the scroll chuck according to Embodiment 1, taken along the line A-A in FIG. 2;

FIG. 4 is a cross sectional view of the retainer for a bone to be processed according to Embodiment 1, taken along the line B-B in FIG. 3;

FIG. 5 is a perspective view of a retainer for a bone to be processed and a scroll chuck according to Embodiment 2;

FIG. 6 is a cross sectional view of the retainer for a bone to be processed and the scroll chuck according to Embodiment 2, taken along the line C-C in FIG. 5;

FIG. 7 is a cross sectional view of a retainer for a bone to be processed and a scroll chuck according to Embodiment 3;

FIG. 8A is a partially exploded side view of a nip member according to Embodiment 3;

FIG. 8B is a cross sectional view of the nip member according to Embodiment 3;

FIG. 8C is a cross sectional view of the nip member according to Embodiment 3, taken along the line D-D in FIG. 8A;

FIG. 9A is a cross sectional view of a retainer for a bone to be processed and a chuck according to an alternative example;

FIG. 9B is a cross sectional view of a retainer for a bone to be processed and a fixture according to an alternative example; and

FIG. 10 is a cross sectional view of a retainer for a bone to be processed and a scroll chuck according to an alternative example.

### DESCRIPTION OF EMBODIMENTS

### Embodiment 1

A retainer for a bone to be processed according to the present embodiment is described below with reference to the drawings. The retainer for a bone to be processed according to the present embodiment retains a bone to be processed that includes a long column shape and in which a through-hole that extends along a longitudinal direction is formed. The retainer for a bone to be processed includes: a base table; and a nip member that nips, together with the base table, the bone to be processed. The base table includes: an inserter that includes a rod shape and a first male screw that is formed on a side wall along a longitudinal direction, and is inserted into the through-hole of the bone to be processed; and a supporter that includes a rod shape, and is linked to the inserter at one end in a longitudinal direction, and of which the area of a cross section that is orthogonal to the longitudinal direction is larger than the area of a cross section of the through-hole, the cross section being orthogonal to the longitudinal direction of the bone to be processed. Moreover, the nip member includes a first screw hole on the inner wall of which a first female screw that is threadedly engaged with the first male screw of the base table is formed, and nips, together with the one end of the supporter of the base table, the bone to be processed in the state of being attached threadedly to the inserter of the base table and abutting on the bone to be processed.

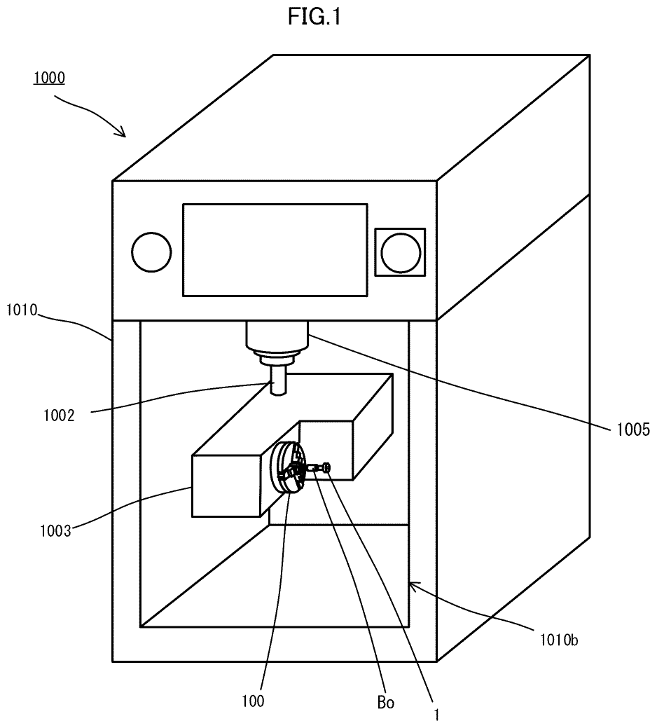

As illustrated in FIG. 1, a cutting-processing device 1000 according to the present embodiment includes: a head 1005 to which a tool 1002 is fixed; and a retaining unit 1003 that is placed to face the head 1005 and retains a bone to be processed Bo. In such a case, the tool 1002 is, for example, a drill for forming a male screw on the bone to be processed Bo. Moreover, the cutting-processing device 1000 includes a housing 1010 that includes a rectangular box shape, in one of the side walls of which an opening 1010b for loading or unloading a retainer for a bone to be processed 1 that retains the bone to be processed Bo is formed, and that houses the head 1005 and the retaining unit 1003 therein. A scroll chuck 100 that retains the retainer for a bone to be processed 1 that retains the bone to be processed Bo is disposed on the retaining unit 1003.

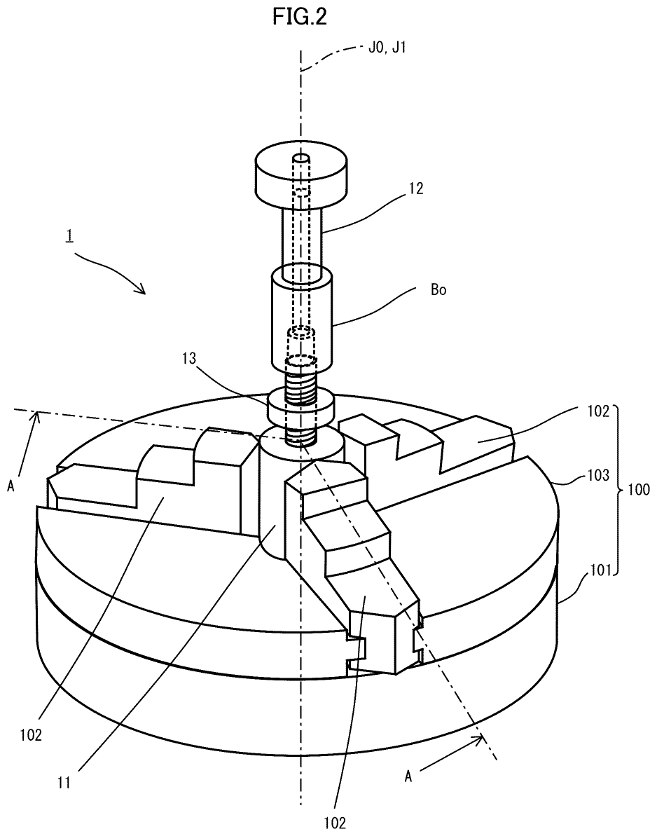

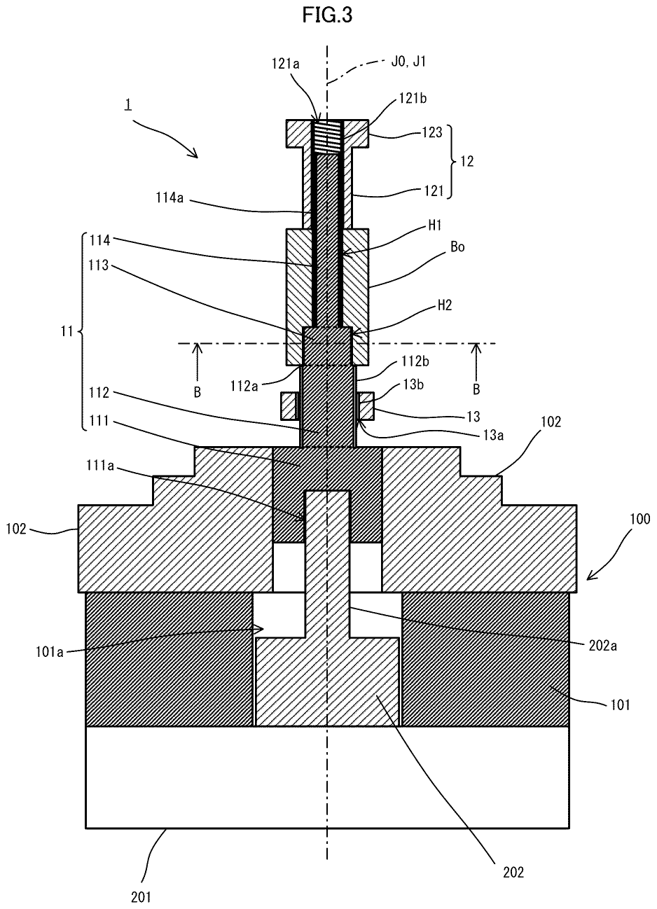

As illustrated in FIG. 2, the scroll chuck 100 can rotate about a rotation axis J0, and is a workpiece retaining device that includes a plurality of (three in FIG. 2) chuck nails 102, a scroll chuck body 103, and a base member 101. The scroll chuck body 103 includes a circular shape in planar view, slidably retains each chuck nail 102 along the diametrical direction of the scroll chuck body 103, and performs centering-chucking of the retainer for a bone to be processed 1 with each chuck nail 102 by simultaneously sliding each chuck nail 102 in the diametrical direction. The bone to be processed Bo includes a long columnar shape. As illustrated in FIG. 3, through-holes H1 and H2 that extend along the longitudinal direction of the bone to be processed Bo are formed in the bone to be processed Bo. A through-hole 101a into which a shaft 202 that is rotated by a motor 201 that rotationally drives the scroll chuck 100 is inserted is formed in the base member 101. The leading end of the shaft 202 is provided with a positioning pin 202a for positioning the retainer for a bone to be processed 1, the positioning pin 202a extending along the rotation axis J0.

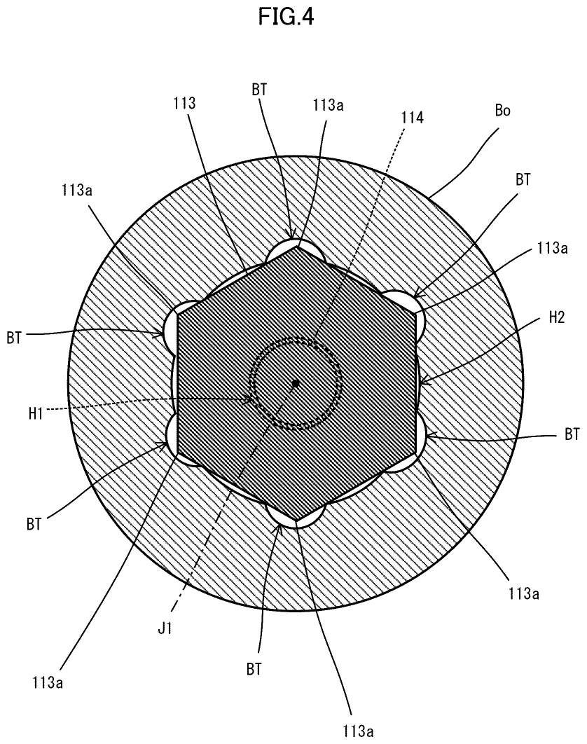

The retainer for a bone to be processed 1 includes: a base table 11; a nip member 12 that nips, together with the base table 11, the bone to be processed Bo; and an extrusion member 13 for extruding the bone to be processed Bo after the processing of the bone to be processed Bo. The base table 11 includes: inserters 113 and 114 that are inserted into the through-holes H1 and H2 of the bone to be processed Bo; a supporter 112 that supports the bone to be processed Bo; and a portion to be retained 111 that is chucked by the scroll chuck 100. The inserter 114 includes a rod shape and a male screw 114a that is formed on a side wall along a longitudinal direction. The inserter 113 is located in a region closer to the portion to be retained 111 in the longitudinal direction of the inserter 114. The inserters 113 and 114 are placed in postures in which the central axis JI along the longitudinal direction thereof coincides with the rotation axis J0 of the scroll chuck 100. The inserter 113 includes a column shape of which a cross section is hexagonal, as illustrated in FIG. 4. Engagement grooves BT with which the six corners 113a of the inserter 113 engage, respectively, are formed on the inner wall of the through-hole H2 of the bone to be processed Bo.

Referring back to FIG. 3, the supporter 112 includes a columnar rod shape, and is linked to the inserter 113 at one end in a longitudinal direction. The area of a cross section of the supporter 112, the cross section being orthogonal to the longitudinal direction of the supporter 112, is larger than the area of a cross section of the through-hole H2 of the bone to be processed Bo, the cross section being orthogonal to the longitudinal direction of the bone to be processed Bo. Thus, a stage 112a on which an edge of the through-hole H2 of the bone to be processed Bo is locked is formed between the inserter 113 and the supporter 112. A male screw 112b is formed on a side wall along the longitudinal direction of the supporter 112.

The portion to be retained 111 includes a columnar shape of which the outer diameter is longer than the outer diameter of the supporter 112, and is linked to another end of the supporter 112, the other end being opposite to a side closer to the inserter 113. The portion to be retained 111 is subjected to centering-chucking by the plurality of chuck nails 102 of the scroll chuck 100 described above. Moreover, a recess 111a into which the leading end of the positioning pin 202a of the shaft 202 described above is fitted is formed on an end surface of the portion to be retained 111, the end surface being opposite to a side closer to the supporter 112.

The nip member 12 includes: a body 121 including a screw hole 121a on the inner wall of which a female screw 121b that is threadedly engaged with the male screw 114a of the inserter 114 is formed; and an outer collar 123 that outwardly overhangs from one end in the extending direction of the screw hole 121a of the body 121 in a direction that is orthogonal to the extending direction. The nip member 12 nips, together with the stage 112a formed on one end, closer to the inserter 113, of the supporter 112, the bone to be processed Bo in the state of being attached threadedly to the inserter 114 of the base table 11 and abutting on the bone to be processed Bo.

The extrusion member 13 is, for example, a nut, and includes a screw hole 13a on which a female screw 13b that is threadedly engaged with the male screw 112b of the supporter 112 is formed. The bone to be processed Bo can be extruded toward the leading end of the inserter 114 of the base table 11 by rotating the extrusion member 13 about the central axis of the supporter 112 along the longitudinal direction in a state in which the extrusion member 13 is allowed to abut on the bottom surface of the bone to be processed Bo after the processing of the bone to be processed Bo.

Subsequently, a step of producing a so-called set screw using the retainer for a bone to be processed 1 according to the present embodiment is described. First, the bone to be processed Bo that is retained by the retainer for a bone to be processed 1 is produced. Specifically, first, a bone chip including a columnar shape is collected from a patient using, for example, a cylindrically-shaped cutting tool. Then, the through-holes (H1 and H2 in FIG. 3) that extend in a direction that is orthogonal to a circular cross section of the taken bone chip are drilled in the bone chip. Then, the engagement grooves BT into which the corners 113a of the inserter 113 fit are formed. The bone to be processed Bo is produced in such a manner.

Then, the bone to be processed Bo is inserted into the inserters 113 and 114 of the base table 11. In such a case, the engagement grooves BT of the bone to be processed Bo mate with the corners 113a of the inserter 113. As a result, the bone to be processed Bo can be prevented from relatively rotationally moving about the rotation axis J0 with respect to the inserters 113 and 114 in the case of cutting-processing of the bone to be processed Bo. Subsequently, the bone to be processed Bo is nipped between the nip member 12 and the supporter 112 of the base table 11 by threadedly engaging the nip member 12 with the inserter 114 to allow the nip member 12 to abut on the end of the bone to be processed Bo. In such a case, the outer diameter of the outer collar 123 of the nip member 12 is set to be smaller than the inner diameter of the tool 1002 (see FIG. 1) such as, for example, a drill for forming the male screw in the bone to be processed Bo, to prevent the outer collar 123 from interfering with the tool 1002. Then, the leading end of the positioning pin 202a of the scroll chuck 100 is fitted into the recess 111a of the portion to be retained 111 in the retainer for a bone to be processed 1 that retains the bone to be processed Bo. Then, the portion to be retained 111 is retained by the chuck nails 102 of the scroll chuck 100. Variations in the retaining position of the bone to be processed Bo in the case of repeating the cutting-processing of the bone to be processed Bo can be suppressed by allowing the scroll chuck 100 to retain the portion to be retained 111 in the state of fitting the leading end of the positioning pin 202a of the scroll chuck 100 into the recess 111a of the portion to be retained 111 of the retainer for a bone to be processed 1 in such a manner. Subsequently, the cutting-processing (thread-cutting processing) is performed by allowing the tool 1002 to approach and come into contact with the bone to be processed Bo from one direction along the rotation axis J0 of the scroll chuck 100 while rotating the scroll chuck 100. In such a manner, a so-called set screw is produced.

If the length of the bone to be processed Bo is short in a case in which the bone to be processed Bo is gripped by the scroll chuck 100, a so-called grip section is unable to be sufficiently secured, and retaining power is reduced. Therefore, the bone to be processed Bo may fall off the scroll chuck 100 in the processing of the bone to be processed Bo. A case in which a bone chip is collected from a part of the bone of a patient to produce, for example, a screw for treating bone fracture is preferred from the viewpoint of reducing a burden on the patient by reducing the amount of the collected bone chip due to the smaller size of the bone to be processed Bo as the base of the screw. In contrast, in accordance with the retainer for a bone to be processed 1 according to the present embodiment, the base table 11 includes: the inserters 113 and 114 that are inserted into the through-holes H1 and H2 of the bone to be processed Bo; and the supporter 112 of which the area of a cross section that is orthogonal to the longitudinal direction is larger than the area of a cross section of the through-hole H2 of the bone to be processed Bo, the cross section being orthogonal to the longitudinal direction of the bone to be processed Bo. The nip member 12 nips, together with the supporter 112, the bone to be processed Bo in the state of being attached threadedly to the inserter 114 of the base table 11 and abutting on the bone to be processed Bo. As a result, the bone to be processed Bo can be firmly retained without directly gripping the side wall of the bone to be processed Bo. Accordingly, the processing accuracy of the cutting-processing of the bone to be processed Bo can be stabilized, and the bone to be processed Bo does not require a portion corresponding to a so-called grip section. Therefore, the amount of the bone to be processed Bo to be collected from a patient can be reduced corresponding thereto, and therefore, a burden on the patient can be reduced.

In a case in which a so-called set screw on the whole side wall of which a male screw is formed is produced as a screw, it is necessary that, first, the male screw is formed on the other portion than a portion, gripped by the scroll chuck 100, in the bone to be processed Bo, the portion, on which the male screw is not formed, in the bone to be processed Bo is then gripped again in the form of exposure thereof, and cutting-processing of the unprocessed portion is performed. Thus, it is impossible to complete cutting-processing of the bone to be processed Bo in one step. When the bone to be processed Bo is gripped again, the chuck nails 102 of the scroll chuck 100 come into contact with the male screw, and therefore, the male screw may also be chipped. In contrast, in accordance with the retainer for a bone to be processed 1 according to the present embodiment, the above-described set screw can be produced by cutting-processing without gripping the bone to be processed Bo again, and therefore, shortening of tact time due to the decreased number of steps and improvement in yield due to prevention of chipping of the male screw in the cutting-processing of the bone to be processed Bo can be achieved.

The inserter 113 of the base table 11 according to the present embodiment includes a hexagonal cross section orthogonal to the longitudinal direction of the inserter 114. The engagement grooves BT with which the corners 113a of the inserter 113 engage are formed on the inner wall of the through-hole H2 of the bone to be processed Bo. As a result, the rotation of the bone to be processed Bo about the central axis J1 is restricted, and therefore, the accuracy of the processing of the bone to be processed Bo can be enhanced.

The engagement grooves BT of the bone to be processed Bo may bite part of the inserter 113 due to a torque load generated in the circumferential direction of the bone to be processed Bo in the processing of the bone to be processed Bo. In such a case, the bone to be processed Bo may be prevented from being smoothly removed from the retainer for a bone to be processed 1 after the processing of the bone to be processed Bo. In such a case, the bone to be processed Bo may be chipped by, for example, grasping the bone to be processed Bo by the hand to intend the bone to be processed Bo to be forcibly pulled out of the inserter 113. In contrast, in the supporter 112 of the base table 11 according to the present embodiment, the male screw 112b is formed on the side wall along the longitudinal direction of the supporter 112. The extrusion member 13 is attached threadedly to the male screw 112b of the supporter 112. As a result, the extrusion member 13 is allowed to abut on the bottom surface of the bone to be processed Bo after the processing of the bone to be processed Bo, and the extrusion member 13 is rotated about the central axis along the longitudinal direction of the supporter 112, whereby a relatively strong force can be generated in the direction of extruding the bone to be processed Bo without exerting a forcible force on the bone to be processed Bo. Accordingly, even in a case in which part of the inserter 113 becomes in the state of biting the engagement grooves BT of the bone to be processed Bo, the state can be canceled without exerting a forcible force on the bone to be processed Bo, and therefore, the bone to be processed Bo can be inhibited from being chipped.

The base table 11 according to the present embodiment is linked to the supporter 112, and includes the portion to be retained 111 that is subjected to centering-chucking by the plurality of chuck nails 102 of the scroll chuck 100. As a result, the bone to be processed Bo can be fixed to the scroll chuck 100 via the retainer for a bone to be processed 1, and therefore, the range of the variations of the cutting-processing device 1000 that can be applied to the cutting-processing of the bone to be processed Bo can be increased.

### Embodiment 2

A retainer for a bone to be processed according to the present embodiment differs from the retainer for a bone to be processed according to Embodiment 1 in that the retainer for a bone to be processed includes coolant suppliers that are disposed in a base table, and through which a coolant is supplied to a side wall of a bone to be processed to thereby cool the bone to be processed in a state in which the bone to be processed is nipped between a nip member and the supporter of the base table.

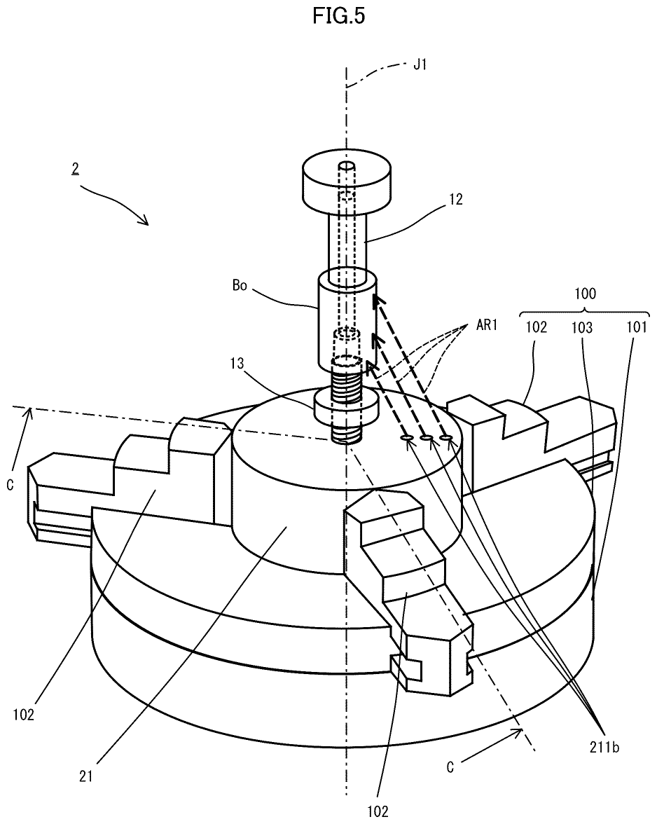

As illustrated in FIG. 5, the retainer for a bone to be processed 2 according to the present embodiment includes: a base table 21; a nip member 12 that nips, together with the base table 21, a bone to be processed Bo; and an extrusion member 13. In FIG. 5, configurations similar to those in Embodiment 1 are denoted by the same reference characters as those in FIG. 2. As indicated by arrows AR1, discharge holes 211b that are the coolant suppliers through which the coolant is discharged toward the side wall of the bone to be processed Bo to thereby supply the coolant to the side wall of the bone to be processed Bo are disposed in the base table 21. For example, physiological saline is adopted as the coolant.

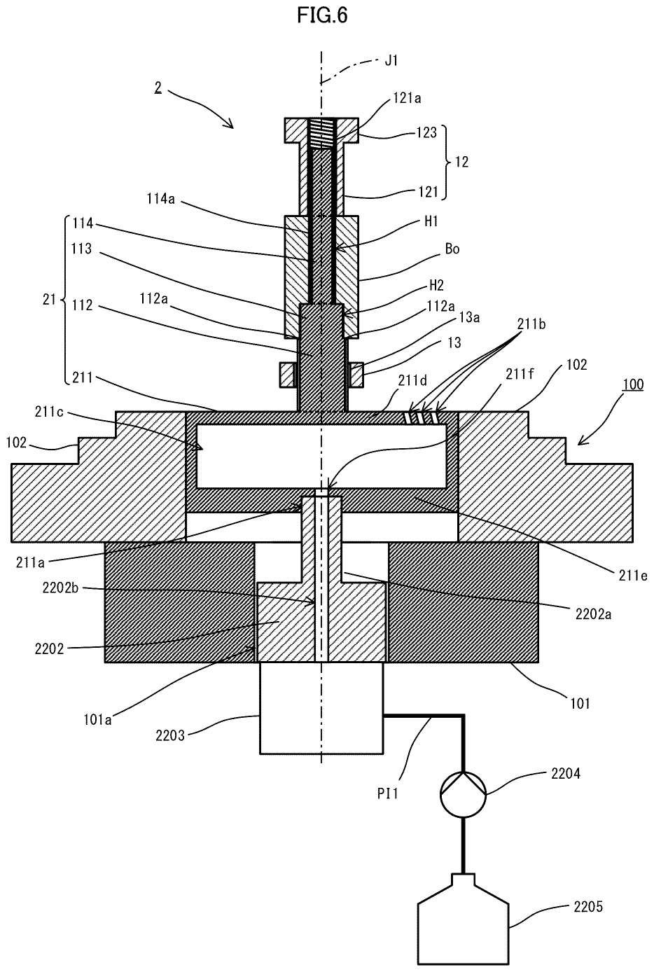

As illustrated in FIG. 6, the base table 21 includes: inserters 113 and 114 that are inserted into the through-holes H1 and H2 of the bone to be processed Bo; a supporter 112 that supports the bone to be processed Bo; and a portion to be retained 211 that is chucked by a scroll chuck 100. In FIG. 6, the same configurations as those in Embodiment 1 are denoted by the same reference characters as those in FIG. 3. The portion to be retained 211 includes a box shape that is circular in planar view, the outer diameter of the shape being larger than the outer diameter of the supporter 112, and includes a storage region 211c in which the coolant is stored. The discharge holes 211b described above are disposed in the top wall 211d, closer to the supporter 112 of the portion to be retained 211. A recess 211a in which the leading end of a positioning pin 2202a that is disposed in the leading end of a shaft 2202 is fitted is formed in the outer surface of the bottom wall 211e of the portion to be retained 211. The bottom of the recess 211a is perforated with a through-hole 211f that communicates with the storage region 211c. For example, an oil seal (not illustrated) is interposed between the inner wall of the recess 211a and the leading end of the positioning pin 2202a, to maintain watertightness.

In such a case, a supply passage 2202b for supplying the coolant into the storage region 211c of the portion to be retained 211 is formed in the shaft 2202 and the positioning pin 2202a. The supply passage 2202b is connected to a pump 2204 through a rotary joint 2203 and a supply pipe PI1. The pump 2204 supplies the coolant that is stored in a coolant storage tank 2205 to the storage region 211c of the portion to be retained 211 through the supply pipe PI1, the rotary joint 2203, and the supply passage 2202b.

In the cutting-processing of the bone to be processed Bo using the retainer for a bone to be processed 2 according to the present embodiment, the cutting-processing of the bone to be processed Bo is performed while supplying the coolant from the discharge holes 211b to the side wall of the bone to be processed Bo in a state in which the bone to be processed Bo is nipped between the nip member 12 and the supporter 112 of the base table 21. In such a case, heat that is generated in the case of the cutting-processing of the bone to be processed Bo is lower than heat that is generated in the case of cutting-processing of, for example, a metal, and therefore, an increase in the temperature of the bone to be processed Bo can be suppressed even by simply discharging a relatively small amount of coolant. In contrast, when the amount of the coolant that is supplied to the side wall of the bone to be processed Bo in the case of the cutting-processing of the bone to be processed Bo is excessively large, a tissue forming the bone to be processed Bo may be conversely damaged. Accordingly, the amount of the coolant that is supplied from the discharge holes 211b to the side wall of the bone to be processed Bo is preferably set at an amount within a previously set range in which the necrosis of the tissue due to the heat of the bone to be processed Bo and the damage to the tissue due to the excessive supply of the coolant can be prevented.

In the cutting-processing of the bone to be processed Bo, the temperature of the bone to be processed Bo is increased due to heat generated when the bone to be processed Bo is cut, and the tissue forming the bone to be processed Bo may necrotize particularly when the temperature of the bone to be processed Bo is increased to 50° C. or more. In contrast, the discharge holes 211b through which the coolant is supplied to the side wall of the bone to be processed Bo in a state in which the bone to be processed Bo is nipped between the nip member 12 and the supporter 112 of the base table 21 are disposed in the base table 21 of the retainer for a bone to be processed 2 according to the present embodiment. As a result, the cutting-processing of the bone to be processed Bo can be performed while suppressing an increase in the temperature of the bone to be processed Bo, and therefore, the necrosis of the tissue forming the bone to be processed Bo can be suppressed.

### Embodiment 3

A retainer for a bone to be processed according to the present embodiment differs from the retainer for a bone to be processed according to Embodiment 1 in view of the structure of a nip member. The nip member according to the present embodiment includes: a body including a first screw hole in which a female screw that is threadedly engaged with the male screw of the inserter of a base table is formed; a first clutch that is placed in a state in which a rotation axis coincides with the central axis of the first screw hole; a second clutch that engages with the first clutch; and a gripper that is fixed to the second clutch. The second clutch idles with respect to the first clutch in a case in which torque at which the first clutch is rotated about the above-described rotation axis by the gripper is more than reference torque.

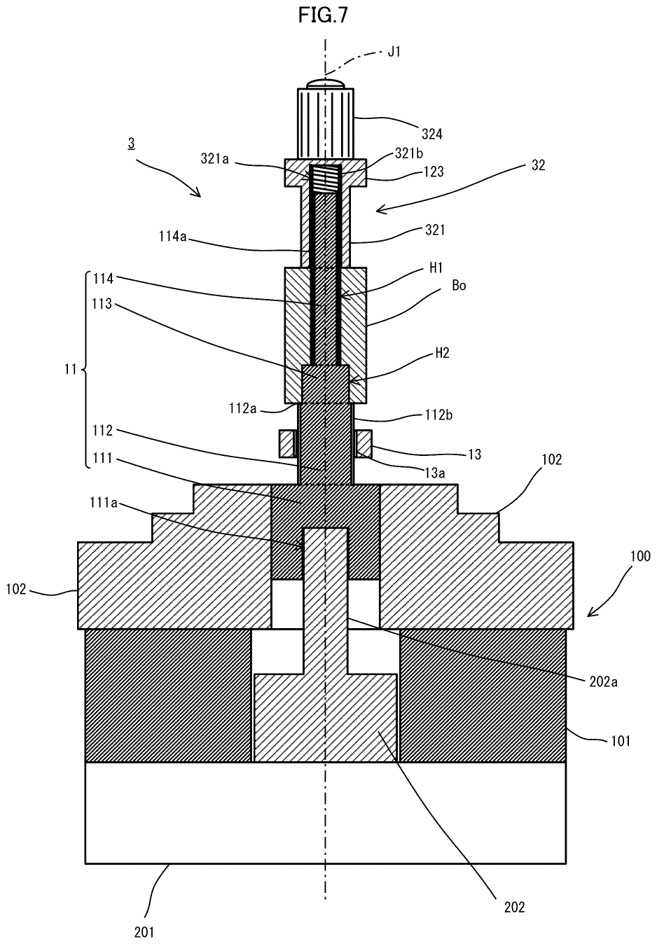

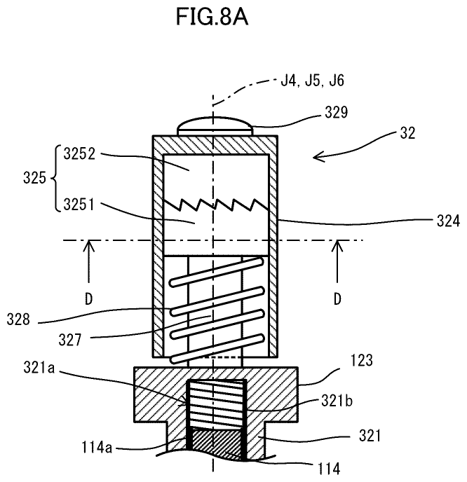

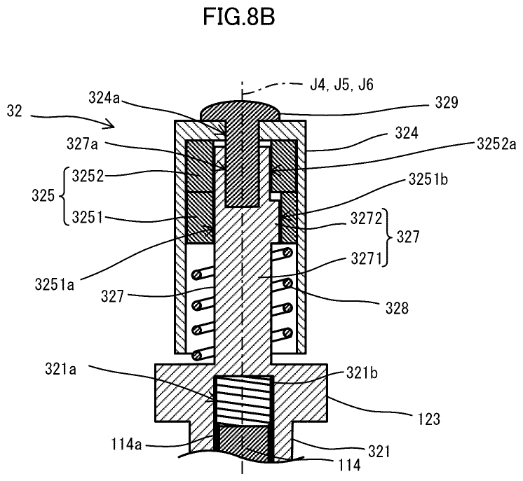

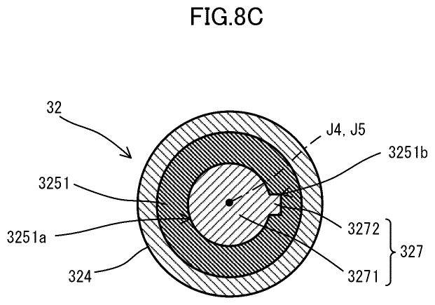

As illustrated in FIG. 7, the retainer for a bone to be processed 3 according to the present embodiment includes: the base table 11; the nip member 32 that nips, together with the base table 11, the bone to be processed Bo; and an extrusion member 13. In FIG. 7, the same configurations as those in Embodiment 1 are denoted by the same reference characters as those in FIG. 2. As illustrated in FIGS. 8A and 8B, the nip member 32 includes a body 321, an outer collar 123, a gripper 324, a claw-clutch 325, a supporter 327, a biasing member 328, and a support member 329. The body 321 includes a columnar shape, and includes a screw hole 321a in which a female screw 321b that is threadedly engaged with the male screw 114a of the inserter 114 of the base table 11 is formed. The supporter 327 includes a long columnar shape, and includes: a supporter body 3271 that is linked to and formed integrally with the body 321, and in which a recess 327a into which part of the support member 329 is fitted and fixed is formed in another end opposite to one end that is linked to the body 321 in a longitudinal direction; and a protrusion 3272 that protrudes, in a diametrical direction, from a portion on the side wall of the supporter body 3271, the portion facing the claw-clutch 325.

The claw-clutch 325 includes: a first clutch member 3251 that is placed in the gripper 324 and perforated with a through-hole 3251a into which the supporter 327 is inserted; and a second clutch member 3252 that is perforated with a through-hole 3252a into which the supporter 327 is inserted. The first clutch member 3251 is disk-shaped, and a plurality of engaging teeth is disposed on one side of the first clutch member 3251 in a thickness direction along a circumferential direction. The rotation axis J4 of the first clutch member 3251 coincides with the central axis J5 of the screw hole 321a of the body 321, and the first clutch member 3251 is placed in a state in which the first clutch member 3251 can be moved in the direction of the rotation axis J4. A groove 3251b that extends in the thickness direction of the first clutch member 3251 and into which the protrusion 3272 of the supporter 327 fits is formed on the inner wall of the through-hole 3251a of the first clutch member 3251. The protrusion 3272 of the supporter 327 is fitted into the groove 3251b, as illustrated in FIG. 8C. As a result, the rotation of the first clutch member 3251 about the rotation axis J4 with respect to the body 321 and the supporter 327 is restricted.

The second clutch member 3252 is disk-shaped, and a plurality of engaging teeth is disposed on one side of the second clutch member 3252 in a thickness direction along a circumferential direction. In such a case, the second clutch member 3252 is placed in a posture in which a rotation axis J6 coincides with the rotation axis J4 of the first clutch member 3251, and the one side on which the plurality of engaging teeth is disposed faces the side on which the plurality of engaging teeth of the first clutch member 3251 is disposed. The engaging teeth of the second clutch member 3252 engage with the plurality of engaging teeth of the first clutch member 3251, whereby the second clutch member 3252 is fastened to the first clutch member 3251.

The biasing member 328 is, for example, a coil spring that is wound around the supporter 327, and biases the first clutch member 3251 in the direction of pressing the first clutch member 3251 against the second clutch member 3252. The gripper 324 includes a bottomed cylindrical shape, and an opening 324a into which a part of the support member 329 is inserted is disposed in the bottom wall of the gripper 324. The gripper 324 is pivoted by the support member 329 that is inserted into the opening 324a. The gripper 324 is fixed to the second clutch member 3252 that is placed in the gripper 324, and can be rotated, together with the second clutch member 3252, around the rotation axis J6 of the second clutch member 3252.

In such a case, when a user grips and rotates the gripper 324 in a state in which the first clutch member 3251 and the second clutch member 3252 are fastened to each other, the supporter 327 and the body 321 are thus rotated about the central axis J5 of the screw hole 321a of the body 321. As a result, the nip member 32 is moved in a direction toward the bone to be processed Bo, and the nip member 32 can be allowed to abut on the bone to be processed Bo. The rotational torque of the second clutch member 3252 is increased when the gripper 324 is further rotated in a state in which the nip member 32 abuts on the bone to be processed Bo. When torque at which the second clutch member 3252 is rotated about the rotation axis J6 by the gripper 324 is more than previously set reference torque, the second clutch member 3252 idles with respect to the first clutch member 3251. In such a case, the reference torque is defined by a force that presses the first clutch member 3251 against the second clutch member 3252 by the biasing member 328. As a result, a force that is applied in the direction of pressing the nip member 32 against the bone to be processed Bo can be restricted in a state in which the nip member 32 abuts on the bone to be processed Bo.

As described above, in accordance with the retainer for a bone to be processed 3 according to the present embodiment, when the torque at which the second clutch member 3252 is rotated about the rotation axis J6 by the gripper 324 is more than the previously set reference torque, the second clutch member 3252 idles with respect to the first clutch member 3251, and the force that is applied in the direction of pressing the nip member 32 against the bone to be processed Bo can be restricted in a state in which the nip member 32 abuts on the bone to be processed Bo. As a result, the magnitude of nipping force at which the bone to be processed Bo is nipped between the nip member 32 and the supporter 112 of the base table 11 can be restricted to magnitude corresponding to the reference torque, and therefore, the breakage of the bone to be processed Bo due to nipping of the bone to be processed Bo at a force that is more than required can be suppressed.

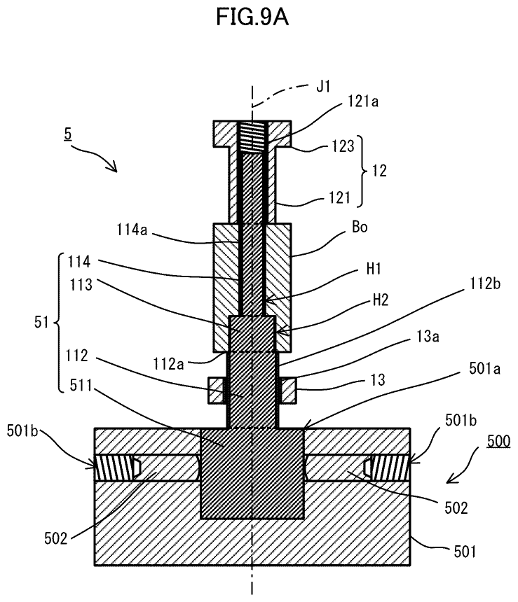

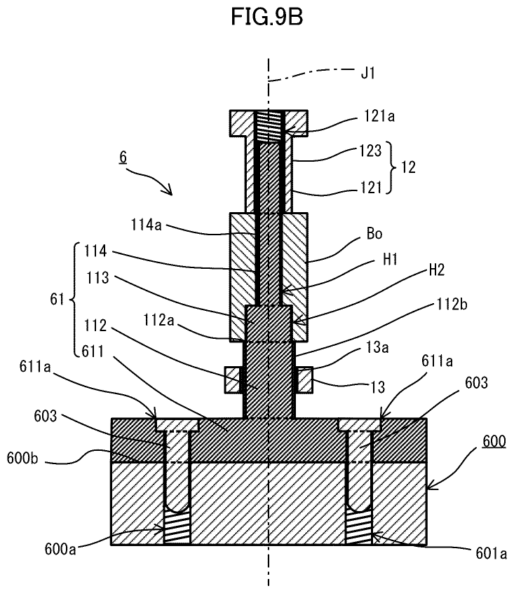

The embodiments of the present disclosure have been described above. However, the present disclosure is not limited to the configurations of the embodiments described above. For example, a case in which a retainer for a bone to be processed 5 is retained by a chuck 500, as illustrated in FIG. 9A, is also acceptable. In FIG. 9A, configurations similar to those in Embodiment 1 are denoted by the same reference characters as those in FIG. 3. The chuck 500 includes a block shape, and includes: a chuck body 501 in which a recess 501a in which a part of the retainer for a bone to be processed 5 is placed and at least one screw hole 501b that communicates with the interior of the recess 501a from an outer wall are formed; and a chuck screw 502 that is threadedly engaged with at least one screw hole 501b, respectively, and of which the leading end abuts on the part of the retainer for a bone to be processed 5, the part being placed in the recess 501a. The base table 51 of the retainer for a bone to be processed 5 includes a portion to be retained 511 that is fitted into the recess 501a of the chuck 500. The portion to be retained 511 is fixed by the chuck screw 502 in the state of being fitted into the recess 501a of the chuck 500.

Alternatively, a case in which a retainer for a bone to be processed 6 is fixed to a fixture 600 as illustrated in FIG. 9B is also acceptable. In FIG. 9B, configurations similar to those in Embodiment 1 are denoted by the same reference characters as those in FIG. 3. In such a case, the fixture 600 includes, for example, a flat block shape, and perforated with at least one screw hole 600a in a thickness direction. The base table 61 of a retainer for a bone to be processed 5 is plate-shaped, and includes a portion to be fixed 611, in which a screw hole 611a is drilled in a portion corresponding to at least one screw hole 600a in the state of being placed on a mounting surface 600b on which the retainer for a bone to be processed 6 is mounted in the fixture 600. The retainer for a bone to be processed 6 is fixed to the fixture 600 by threadedly engaging a fixation screw 603 that is attached threadedly into the screw hole 611a of the portion to be fixed 611 with the screw holes 600a and 601a of the fixture 600 in a state in which the portion to be fixed 611 of the base table 61 is mounted on the mounting surface 600b of the fixture 600.

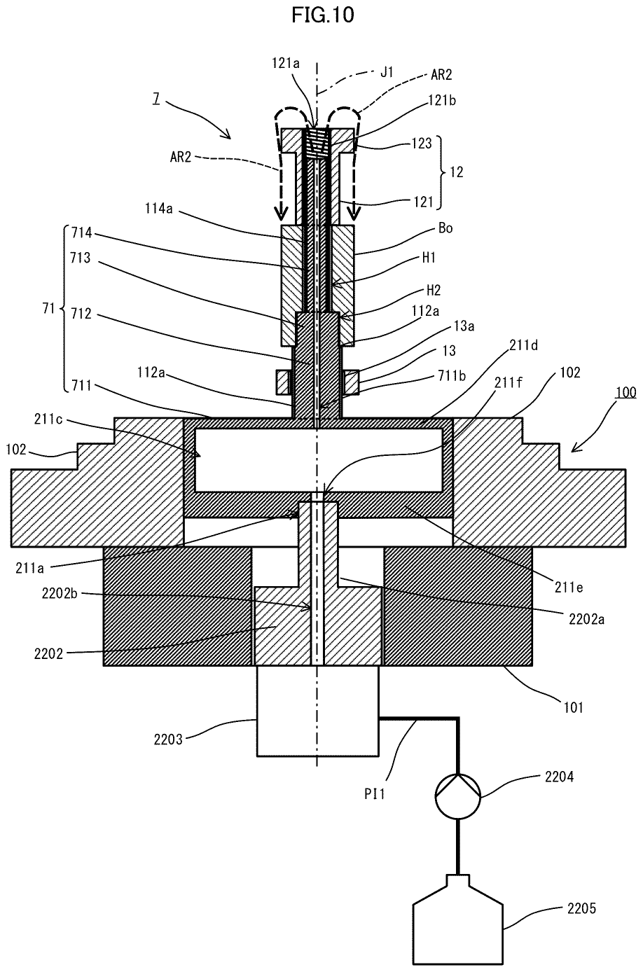

In Embodiment 2, an example of the retainer for a bone to be processed 2 in which the discharge holes 211b through which the coolant is supplied to the side wall of the bone to be processed Bo by discharging the coolant toward the side wall of the bone to be processed Bo is disposed in the base table 21 is described. However, the configuration of the coolant supplier disposed in the retainer for a bone to be processed is not limited thereto. For example, as in the case of a retainer for a bone to be processed 7 illustrated in FIG. 10, a coolant may be allowed to flow out of the leading end of the inserter 714 of a base table 71 and may be supplied to the side wall of a bone to be processed Bo, as indicated by arrows AR2. In FIG. 10, configurations similar to those in Embodiment 2 are denoted by the same reference characters as those in FIG. 8.

In such a case, the retainer for a bone to be processed 7 includes: the base table 71; a nip member 12 that nips, together with the base table 71, the bone to be processed Bo; and an extrusion member 13. The base table 71 includes: inserters 713 and 714 that are inserted into the through-holes H1 and H2 of the bone to be processed Bo; a supporter 712 that supports the bone to be processed Bo; and a portion to be retained 711 that is chucked by a scroll chuck 100. A supply passage 711b for supplying a coolant that is stored in a storage region 211c to the leading end of the inserter 714 is formed in the base table 71. In such a case, one end of the supply passage 711b communicates with the storage region 211c of the portion to be retained 711, and the other end of the supply passage 711b communicates with the interior of the screw hole 121a of the nip member 12 in the leading end of the inserter 714.

In each embodiment, an example in which the bone to be processed Bo is collected from part of the bone of a patient has been described. However, the bone to be processed Bo is not limited thereto, but may be formed of an artificial bone material such as, for example, hydroxyapatite/poly(L-lactic acid) composite (HA-PLLA). A workpiece that is formed of a rare metal such as tungsten or an expensive material such as diamond can also be applied instead of the bone to be processed Bo.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

This application claims the benefit of Japanese Patent Application No. 2021-180966, filed on Nov. 5, 2021, the entire disclosure of which is incorporated by reference herein.

### Industrial Applicability

The present disclosure is suitable as a retainer for a bone to be processed that is used in a cutting-processing device that performs cutting-processing of the bone.

### REFERENCE SIGNS LIST

1, 2, 3, 5, 6, 7 Retainer for bone to be processed
11, 21, 51, 61, 71 Base table
12, 32 Nip member
13 Extrusion member
13a, 121a, 321a, 501b, 600a, 601a, 611a Screw hole
13b, 121b, 321b Female screw
100 Scroll chuck
101 Base member
101a, 211f, 3251a, 3252a, H1, H2 Through-hole
102 Chuck nail
103 Scroll chuck body
111, 211, 511, 711 Portion to be retained
111a, 211a, 327a, 501a Recess
112, 327, 712 Supporter
112a Stage
112b, 114a Male screw
113, 114, 713, 714: Inserter
113a Corner
121, 321 Body
123 Outer collar
201 Motor
202, 2202 Shaft
202a, 2202a Positioning pin
211b Discharge hole
211c Storage region
211d Top wall
211e Bottom wall
324 Gripper
324a, 1010b Opening
325 Claw-clutch
327a Recess
328 Biasing member
329 Support member
500 Chuck
501 Chuck body
502 Chuck screw
600 Fixture
600b Mounting surface
603 Fixation screw
611 Portion to be fixed
711b, 2202b Supply passage
1000 Cutting-processing device
1002 Tool
1003 Retaining unit
1005 Head
1010 Housing
2203 Rotary joint
2204 Pump
2205 Coolant storage tank
3251 First clutch member
3251b Groove
3252 Second clutch member
3271 Supporter body
3272 Protrusion
Bo Bone to be processed
BT Engagement groove
J0, J4, J6 Rotation axis
J1, J5 Central axis
PI1 Supply pipe

1. A retainer for a bone to be processed that retains a bone to be processed that having a long column shape and in which a through-hole that extends along a longitudinal direction is formed, the retainer for a bone to be processed comprising:
a base table comprising: an inserter that has a rod shape and a first male screw that is formed on a side wall along a longitudinal direction, and is inserted into the through-hole; and a supporter that has a rod shape, is linked to the inserter at one end in a longitudinal direction, and supports the bone to be processed, and of which an area of a cross section that is orthogonal to the longitudinal direction is larger than an area of a cross section of the through-hole, the cross section being orthogonal to the longitudinal direction of the bone to be processed; and
a nip member that has a first screw hole on an inner wall of which a first female screw that is threadedly engaged with the first male screw is formed, and nips, together with the one end of the supporter, the bone to be processed in a state of being attached threadedly to the inserter and abutting on the bone to be processed.

2. The retainer for a bone to be processed according to claim 1, wherein
the inserter comprises at least one protrusion that protrudes in a direction of leaving a central axis along the longitudinal direction of the inserter in a direction that is orthogonal to the longitudinal direction of the inserter at another end in the longitudinal direction of the inserter, and
an engagement groove that is formed on an inner wall of the through-hole in at least one of both ends in an extending direction of the through-hole, and with which the at least one protrusion engages is formed in the bone to be processed.

3. The retainer for a bone to be processed according to claim 1, wherein
in the supporter, a second male screw is formed on a side wall along the longitudinal direction of the supporter, and
the retainer for a bone to be processed further comprises an extrusion member that has a second screw hole of an inner wall of which a second female screw that is threadedly engaged with the second male screw is formed, and extrudes the bone to be processed by rotating the extrusion member about a central axis along the longitudinal direction of the supporter in a state of being attached threadedly to the supporter and abutting on the bone to be processed.

4. The retainer for a bone to be processed according to claim 1, wherein
the retainer for a bone to be processed further comprises a coolant supplier that is disposed in the base table, and through which a coolant is supplied to a side wall of the bone to be processed to thereby cool the bone to be processed in a state in which the bone to be processed is nipped between the nip member and the one end of the supporter.

5. The retainer for a bone to be processed according to claim 1, wherein
the nip member comprises:
a body having the first screw hole;
a first clutch member of which a rotation axis coincides with a central axis of the first screw hole and rotation about the rotation axis is restricted with respect to the body, and that is placed in a state in which the first clutch member can be moved in a direction of the rotation axis;
a second clutch member of which rotation axis coincides with the rotation axis of the first clutch member, and that is fastened to the first clutch member;
a biasing member that biases the first clutch member in a direction of pressing the first clutch member against the second clutch member; and
a gripper that is fixed to the second clutch member, and
the second clutch member idles with respect to the first clutch member when torque at which the first clutch member is rotated about the rotation axis by the gripper is more than reference torque that is defined by a force that presses the first clutch member against the second clutch member by the biasing member.

6. The retainer for a bone to be processed according to claim 1, wherein the base table is linked to another end of the supporter, and further comprises a portion to be retained that is subjected to centering-chucking by a plurality of the chuck nails of a workpiece retaining device comprising the plurality of chuck nails.

7. The retainer for a bone to be processed according to claim 2, wherein
in the supporter, a second male screw is formed on a side wall along the longitudinal direction of the supporter, and
the retainer for a bone to be processed further comprises an extrusion member that has a second screw hole of an inner wall of which a second female screw that is threadedly engaged with the second male screw is formed, and extrudes the bone to be processed by rotating the extrusion member about a central axis along the longitudinal direction of the supporter in a state of being attached threadedly to the supporter and abutting on the bone to be processed.

8. The retainer for a bone to be processed according to claim 2, wherein
the retainer for a bone to be processed further comprises a coolant supplier that is disposed in the base table, and through which a coolant is supplied to a side wall of the bone to be processed to thereby cool the bone to be processed in a state in which the bone to be processed is nipped between the nip member and the one end of the supporter.

9. The retainer for a bone to be processed according to claim 3, wherein
the retainer for a bone to be processed further comprises a coolant supplier that is disposed in the base table, and through which a coolant is supplied to a side wall of the bone to be processed to thereby cool the bone to be processed in a state in which the bone to be processed is nipped between the nip member and the one end of the supporter.

10. The retainer for a bone to be processed according to claim 7, wherein
the retainer for a bone to be processed further comprises a coolant supplier that is disposed in the base table, and through which a coolant is supplied to a side wall of the bone to be processed to thereby cool the bone to be processed in a state in which the bone to be processed is nipped between the nip member and the one end of the supporter.

11. The retainer for a bone to be processed according to claim 2, wherein
the nip member comprises:
a body having the first screw hole;
a first clutch member of which a rotation axis coincides with a central axis of the first screw hole and rotation about the rotation axis is restricted with respect to the body, and that is placed in a state in which the first clutch member can be moved in a direction of the rotation axis;
a second clutch member of which rotation axis coincides with the rotation axis of the first clutch member, and that is fastened to the first clutch member;
a biasing member that biases the first clutch member in a direction of pressing the first clutch member against the second clutch member; and
a gripper that is fixed to the second clutch member, and
the second clutch member idles with respect to the first clutch member when torque at which the first clutch member is rotated about the rotation axis by the gripper is more than reference torque that is defined by a force that presses the first clutch member against the second clutch member by the biasing member.

12. The retainer for a bone to be processed according to claim 3, wherein
the nip member comprises:
a body having the first screw hole;
a first clutch member of which a rotation axis coincides with a central axis of the first screw hole and rotation about the rotation axis is restricted with respect to the body, and that is placed in a state in which the first clutch member can be moved in a direction of the rotation axis;
a second clutch member of which rotation axis coincides with the rotation axis of the first clutch member, and that is fastened to the first clutch member;
a biasing member that biases the first clutch member in a direction of pressing the first clutch member against the second clutch member; and
a gripper that is fixed to the second clutch member, and
the second clutch member idles with respect to the first clutch member when torque at which the first clutch member is rotated about the rotation axis by the gripper is more than reference torque that is defined by a force that presses the first clutch member against the second clutch member by the biasing member.

13. The retainer for a bone to be processed according to claim 4, wherein
the nip member comprises:
a body having the first screw hole;
a first clutch member of which a rotation axis coincides with a central axis of the first screw hole and rotation about the rotation axis is restricted with respect to the body, and that is placed in a state in which the first clutch member can be moved in a direction of the rotation axis;
a second clutch member of which rotation axis coincides with the rotation axis of the first clutch member, and that is fastened to the first clutch member;
a biasing member that biases the first clutch member in a direction of pressing the first clutch member against the second clutch member; and
a gripper that is fixed to the second clutch member, and
the second clutch member idles with respect to the first clutch member when torque at which the first clutch member is rotated about the rotation axis by the gripper is more than reference torque that is defined by a force that presses the first clutch member against the second clutch member by the biasing member.

14. The retainer for a bone to be processed according to claim 7, wherein
the nip member comprises:
a body having the first screw hole;
a first clutch member of which a rotation axis coincides with a central axis of the first screw hole and rotation about the rotation axis is restricted with respect to the body, and that is placed in a state in which the first clutch member can be moved in a direction of the rotation axis;
a second clutch member of which rotation axis coincides with the rotation axis of the first clutch member, and that is fastened to the first clutch member;
a biasing member that biases the first clutch member in a direction of pressing the first clutch member against the second clutch member; and
a gripper that is fixed to the second clutch member, and
the second clutch member idles with respect to the first clutch member when torque at which the first clutch member is rotated about the rotation axis by the gripper is more than reference torque that is defined by a force that presses the first clutch member against the second clutch member by the biasing member.

15. The retainer for a bone to be processed according to claim 8, wherein
the nip member comprises:
a body having the first screw hole;
a first clutch member of which a rotation axis coincides with a central axis of the first screw hole and rotation about the rotation axis is restricted with respect to the body, and that is placed in a state in which the first clutch member can be moved in a direction of the rotation axis;
a second clutch member of which rotation axis coincides with the rotation axis of the first clutch member, and that is fastened to the first clutch member;
a biasing member that biases the first clutch member in a direction of pressing the first clutch member against the second clutch member; and
a gripper that is fixed to the second clutch member, and
the second clutch member idles with respect to the first clutch member when torque at which the first clutch member is rotated about the rotation axis by the gripper is more than reference torque that is defined by a force that presses the first clutch member against the second clutch member by the biasing member.

16. The retainer for a bone to be processed according to claim 9, wherein
the nip member comprises:
a body having the first screw hole;
a first clutch member of which a rotation axis coincides with a central axis of the first screw hole and rotation about the rotation axis is restricted with respect to the body, and that is placed in a state in which the first clutch member can be moved in a direction of the rotation axis;
a second clutch member of which rotation axis coincides with the rotation axis of the first clutch member, and that is fastened to the first clutch member;
a biasing member that biases the first clutch member in a direction of pressing the first clutch member against the second clutch member; and
a gripper that is fixed to the second clutch member, and
the second clutch member idles with respect to the first clutch member when torque at which the first clutch member is rotated about the rotation axis by the gripper is more than reference torque that is defined by a force that presses the first clutch member against the second clutch member by the biasing member.

17. The retainer for a bone to be processed according to claim 2, wherein the base table is linked to another end of the supporter, and further comprises a portion to be retained that is subjected to centering-chucking by a plurality of the chuck nails of a workpiece retaining device comprising the plurality of chuck nails.

18. The retainer for a bone to be processed according to claim 3, wherein the base table is linked to another end of the supporter, and further comprises a portion to be retained that is subjected to centering-chucking by a plurality of the chuck nails of a workpiece retaining device comprising the plurality of chuck nails.

19. The retainer for a bone to be processed according to claim 4, wherein the base table is linked to another end of the supporter, and further comprises a portion to be retained that is subjected to centering-chucking by a plurality of the chuck nails of a workpiece retaining device comprising the plurality of chuck nails.

20. The retainer for a bone to be processed according to claim 5, wherein the base table is linked to another end of the supporter, and further comprises a portion to be retained that is subjected to centering-chucking by a plurality of the chuck nails of a workpiece retaining device comprising the plurality of chuck nails.
